(19) United States
(12) Patent Application Publication
(10) Pub. No.: US 2017/0172946 A1
(43) Pub. Date: Jun. 22, 2017

(54) MICRO-SOLID PHASE EXTRACTION

(71) Applicants: Stephan Hau, Skarpnack (SE); Håkan Fischer, Stockholm (SE); Lennart Högman, Stockholm (SE)

(72) Inventors: Stephan Hau, Skarpnack (SE); Håkan Fischer, Stockholm (SE); Lennart Högman, Stockholm (SE)

(21) Appl. No.: 15/386,955

(22) Filed: Dec. 21, 2016

Related U.S. Application Data
(60) Provisional application No. 62/270,402, filed on Dec. 21, 2015.

(51) Int. Cl. A61K 31/137; A61K 9/20

(52) U.S. Cl. CPC A61K 31/137; A61K 9/2095

(57) ABSTRACT

A method of producing a biologic liquid sampling tablet is disclosed and includes molecularly imprinting a polymer over a matrix of an analyte of interest for biological testing; and removing the matrix from the imprinted polymer to form a porous tablet. The tablet is sized to be inserted in an ampoule or human oral cavity.

### CLAIM OF PRIORITY

This application claims priority to U.S. Provisional Application Ser. No. 62/270,402, filed on Dec. 21, 2015, the entire contents of which are hereby incorporated by reference.

### TECHNICAL FIELD

This document generally describes technology related to extracting solid phase material from a liquid sample, such as plasma, urine, or saliva.

### BACKGROUND

Various medical testing operations such as diagnostic testing involve drawing a bodily fluid such as blood, plasma, urine, or saliva, and determining levels of various analytes in the obtained fluid. Solid analytes in a liquid may be extracted to permit such analysis of the analytes. For example, solid-phase microextraction (SPME) uses a fiber coated with an extracting phase (whether a liquid (polymer) or a solid (sorbent)) which extracts different kinds of analytes (including both volatile and non-volatile) from different kinds of media, that can be in liquid or gas phase. The quantity of analyte extracted by the fibre is proportional to its concentration in the sample as long as equilibrium is reached or, in case of short-time pre-equilibrium, with help of convection or agitation. Similarly, stir-bar sorptive extraction (SBSE) is a solventless sample preparation method for extracting and enriching organic compounds from aqueous matrices. The solutes are extracted into a polymer coating on a magnetic stirring rod. The extraction is controlled by the partitioning coefficient of the solutes between the polymer coating and the sample matrix, and by the phase ratio between the polymer coating and the sample volume.

### SUMMARY

This document generally describes materials technology by which a polymer or similar tablet is used for solid-phase extraction in a liquid sample. The tablet is placed in the sample, and the solid phase material passes into the porous tablet until equilibrium is reached. The analyte can then be removed, such as by using ethanol, or can otherwise by introduced into an analysis machine such as at the injection port of a separating instrument, such as a gas chromatography or mass spectrometry machine. The tablet in some examples is formed as a molecularly imprinted polymer (MIP-Tablet) that uses a thin film of polymer, or can be graphitic sorbent (G-Tablet) and silica sorbent (Silica-Tablet) in form. Molecular Imprinting generally involves polymerizing monomers in the presence of a template molecule so that the polymer forms around the template molecule. The template molecule is then extracted, leaving complementary cavities behind. Then when the polymer is introduced to a sample of the same type of molecule, those cavities can fill with such molecule, and not be filled by larger or non-complementary molecules, or filled and readily vacated by much smaller substances in a sample. Such techniques may be used, for example, in the analysis and determination of methadone in blood plasma and amphetamine in urine.

In one implementation, a solid-form sampling tablet is disclosed. The tablet comprises a tablet formed of a polymer having applied to it a thin-film polymer and having a porosity sized to accept a solid-form analyte of interest from a liquid sample and to hold the solid-form analyte in an internal portion of the tablet. The tablet may be sized for oral introduction and holding by a human subject. The tablet may be in the form of a short cylinder, and may be 1 cm or less in diameter, and 0.5 cm or less in height. The tablet may additionally or alternatively contain voids of the same size and shape as the solid-form analyte to which the tablet is directed. Moreover, the tablet can be formed by molecularly imprinting a polymer around a form of the analyte to which the tablet is directed for its testing. Also, the analyte to which the tablet is directed may be selected from the group consisting of methadone and amphetamine.

In another implementation, a method of producing a biologic liquid sampling tablet is disclosed. The method comprises molecularly imprinting a polymer over a matrix of an analyte of interest for biological testing; and removing the matrix from the imprinted polymer to form a porous tablet, wherein the tablet is sized to be inserted in an ampoule or human oral cavity. The method may also comprise inserting the porous tablet in a liquid sample containing the analyte of interest; removing the tablet from the sample containing the analyte of interest; and submitting the analyte captured in the tablet for automated chemical analysis. In some aspects, the liquid sample is inside an oral cavity of a patient to be analyzed. The tablet may be maintained in the oral cavity for a time determine to be sufficient to infuse the tablet with a testable amount of the analyte of interest. Also, submitting the analyte captured in the tablet may comprise removing the analyte from the tablet by subjecting the tablet to a solvent appropriate to remove the analyte from the tablet.

In certain implementations, the systems and techniques discussed here may provide one or more advantages. For example, the techniques discussed here may permit accurate extraction of analytes with relatively high selectivity in small available volumes. Such extraction may occur relatively quickly and efficiently, at a low cost to manufacture the disclosed tablets or other forms of extraction structures.

The details of one or more embodiments are set forth in the accompanying drawings and the description below. Other features and advantages will be apparent from the description and drawings, and from the claims.

### DESCRIPTION OF DRAWINGS

FIG. 1A shows a plurality of extraction tablets in a sample dish.

FIG. 1B shows a single extraction tablet in a small liquid sample.

FIG. 1C shows the tablet formation process in terms of its chemistry.

FIG. 2 is a flow chart of a process for extracting and testing solid-phase material.

FIG. 3 shows a chromatogram for methadone in a plasma sample and blank plasma extracted by a tablet like that shown in FIGS. 1A and 1B.

FIG. 4 is a table that compares LOD, LLOQ extraction time and accuracy for different solid-phase extraction techniques.

Like reference symbols in the various drawings indicate like elements.

### DETAILED DESCRIPTION

This document generally describes techniques for extracting solid-phase material from a fluid sample for purposes of testing the extracted material as an analyte. Such testing can take a variety of familiar forms, and particularly can involve testing for levels of methadone or amphetamine in a patient. The techniques described here focus on the manufacture and use of porous tablets and similar forms made of a molecularly imprinted polymer, carbon material, silica, or sol-gel, and restricted access material (RAM). The developed tablets include voids that match the form of the particular solid analyte that is sought to be captured, by forming the tablet around a sample of such analyte, and then vacating the production-time analyte from the tablet so as to make room for analyte from a testing sample to enter it.

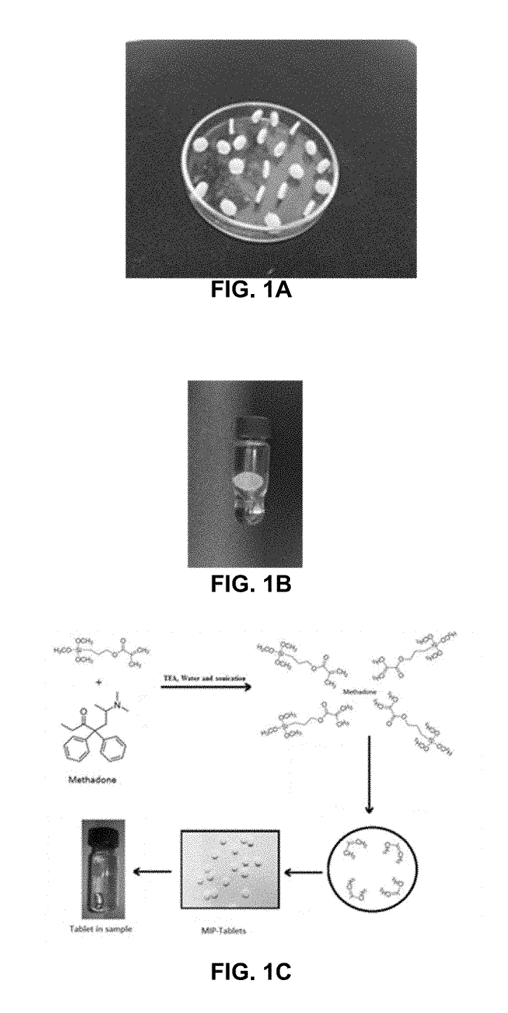

FIG. 1A shows a plurality of extraction tablets in a sample dish, e.g., a petri dish or other liquid-resistant dish that can hold the sample without contamination. The tablets are porous in form and on the order of a cm in diameter and less than a cm thick (e.g., less than 0.5 cm thick). They may be constructed from molecularly-imprinted polymers, carbon material, silica, sol-gel, and restricted access material (RAM). The porosity and internal cavity sizes may be adjusted to be appropriate to desired adsorption capacity and the material to be absorbed—i.e., the internal passages may be sized to accept the solid phase material from outside the tablet and to them hold the material from easily escaping. Such adjustment may be achieved, for example, by forming the form of the tablet around a matrix made up of the analyte that is desired to be tested by the particular tablet. In other words, a first tablet may be indicated as a methadone tablet, while another could be indicated as an amphetamine tablet. A tablet may also have multiple zones, where each zone is formed to absorb a particular analyte, such as a tablet whose left half absorbs methadone as an analyte and whose right side absorbs amphetamine. The solid phase material may then be desorbed by a solvent such as methanol, which may in turn be injected into LC-MS. The material may also be removed by heating the tablet directly into GC-MS. And the tablet may be used for MALDI mass spectrometry. Where the tablet has multiple different zones, it may be cut into pieces at or near the transition area (and a small zone on each side of the transition may be discarded), with each side being subjected to testing independently. Where the analytes are known to not interfere with each other as part of the analysis process, they can both or all be left in the tablet and processed together.

FIG. 1B shows a single extraction tablet in a small liquid sample. Here, the sample is held in a small ampoule so as to make complete immersion of the tablet easier to perform. In other implementations, a caplet-shaped tablet may better fit within the ampoule. In various examples, the sample volume may be relatively small, such as in a range from 100 to 200 micro-liters, suitable for biological fluids from humans and smaller animals such as mice. As noted, the tablet may also be placed in a subject's mouth for an appropriate period where the sample is to be in the form of saliva. The analyte may also be enriched after it is captured, by using, for example, a sample size greater than 200 micro-liters, and then desorbing the analyte into a smaller volume of solvent (e.g., less than 100 micro-liter).

Although a short cylinder tablet is shown in the images, other shapes and sizes of tablet or other forms may be employed in appropriate circumstances. For example, a tubular form (perhaps with rounded ends), such as in the form of a caplet, may be used to provide additional surface area in a form factor that can still be placed easily longitudinally in an ampoule or held in a patient's mouth, and also be seen as a familiar shape by a patient for oral insertion.

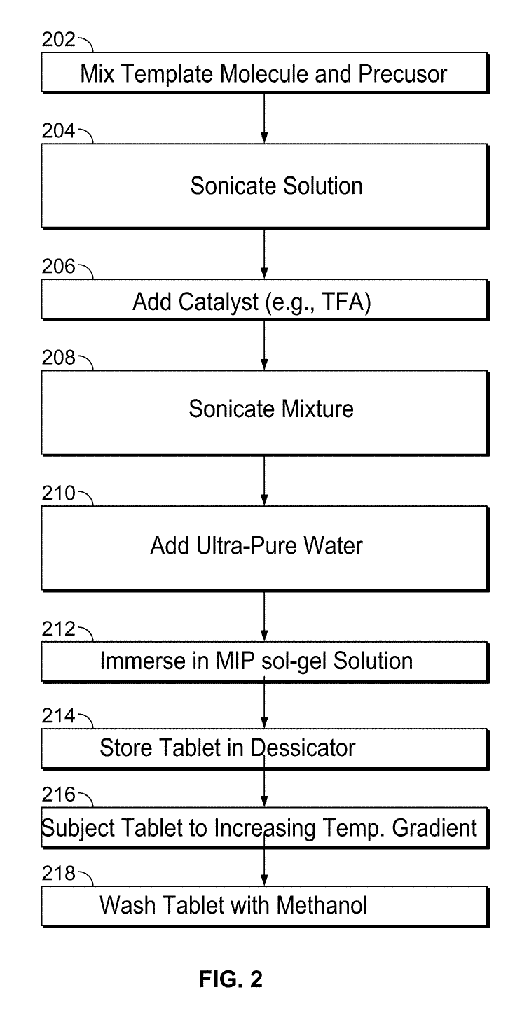

FIG. 1C shows the tablet formation process in terms of its chemistry, and is representative of the process discussed in more detail next with respect to FIG. 2.

FIG. 2 is a flow chart of a process for extracting and testing solid-phase material. In general, the process involves sonicating a relevant solution with a catalyst to form a tablet, and then immersing a prepared tablet in a molecularly imprinted polymer (MIP) sol-gel solution, followed by dessication and poly-condensation at elevated temperature to set the tablet, followed by methanol washing to remove the analyte matrix and make the tablet ready for use. The process may be carried out using an initial liquid material (liquid polymer or sol-gel) such as polyethelene in tablet form as a backbone and a polymer surrounding the polyethylene. The process may also use a powdered starting material such as graphitic, silica, or MIP. A thin film may be applied to the tablet, in particular, for use with gathering saliva samples.

The process begins at step 202, where a solution is prepared that contains a mixture of 0.1 mmol/L template molecule (an analyte of interest) and 3-(propylmethacrylate) trimethoxysilane (used as precursor) in acetonitrile as solvent (400 μL). The analyte of interest may take any of a variety of desired forms, and in the examples discussed here may be methadone or amphetamine.

At box 204, that solution is then sonicated for approximately 30 min. That process agitates the components of the solution and causes them to be evenly dispersed in a relevant pattern within the solution. In this manner, the matrix is evenly dispersed, and the in-polymer pattern that will be created by the matrix will also be evenly dispersed, so as to maximize the performance of the formed tablet.

At box 206, 400 μL of Trifluoroacetic Acid (TFA) is added to the mixture to act as a catalyst. The TFA causes a reaction to occur among the other components of the mixture so that they begin to solidify into the final form for the tablet, around the matrix. Other appropriate solidifying catalysts may also be used, depending on the type of polymer that us used to form the tablet.

At box 208, the resulting mixture is sonicated for approximately 2 min. Such action causes the catalyst to be spread more evenly among the mixture as it works and to catalyze the mixture more evenly throughout the mixture, so that full chemical reaction is performed in the material.

At box 210, approximately 100 μL of milli-Q water (EMD Millipore Corporation, Billerica, Mass.) or other ultra-pure Type 1 water is added. The solution is then kept at room temperature for approximately 30 minutes. During this time, the polymer may better set into its final form.

At box 212, to prepare an imprinted sol-gel layer on both sides of the polyethylene as a tablet form, the material is immersed in the MIP sol-gel solution for 10 min at room temperature, and then placed in a desiccator for 10 min. The step may be repeated, such as two times. The form in this example is 6×1.2 mm, though larger dimensions can be used, consistent with a level of solids that need to be captured for whatever relevant investigation is to be performed using the tablet.

The MIP-Tablet so formed may then be stored in a desiccator for 24 hours or other appropriate time to sufficiently dessicate the material (box 214).

At box 216, for poly-condensation, the MIP-Tablet is subjected to a temperature gradient started at 50 for one minute and increased to 130° C. and then kept at 130° C. 6 hours. Such action finalizes the polymer form for the tablet.

And at box 218, to remove the trapped template and create a porous selective surface, the MIP-Tablet is washed with methanol or other appropriate chemical for removing the template, for 2 hours and with 0.2% formic acid in water for 30 min. The MIP-Tablet in this example is then ready to use, though it may be conditioned with methanol and water before using for plasma or urine matrices.

For such use then, the tablet may be partially or fully submerged in a sample of plasma, urine, saliva, or other appropriate fluid sample. It may be left there for an appropriate period to permit intrusion of the relevant solid-phase component from the sample. The tablet may also be moved or the sample may be stirred or agitated to increase the speed with which the analyte moves into the tablet.

The tablet may then be removed from the sample, or the sample removed from around the tablet, and the tablet may be washed in an appropriate chemical to cause the solid-phase material to exit from the tablet. Such material may then be tested by an appropriate instrument such as a chromatograph, in known manners. Where the sample is saliva, a tablet may be inserted into a test subject's mouth and held there for an appropriate period of time, thereby eliminating other steps from the process of gathering the saliva and isolating solid-form materials from it.

For powdered materials used in such a process (e.g., silica, carbon, or polymer), the materials may be compressed together and added in stainless steel thick tubing with an internal diameter of 5-10 mm, with a tablet prepared under high pressure (ton/in2). Other formation techniques may, in appropriate circumstances, also be used, including extrusion followed by chopping of the extruded column at tablet thickness locations, insertion into tablet-shaped molds, and other appropriate polymer or similar techniques, where the relevant analyte may be included in the material before it hardens into final form so as to create a mold around which the material is formed, and may then be removed by appropriate action such as subjecting the combination to a solvent that is effective on the analyte but not on the tablet itself.

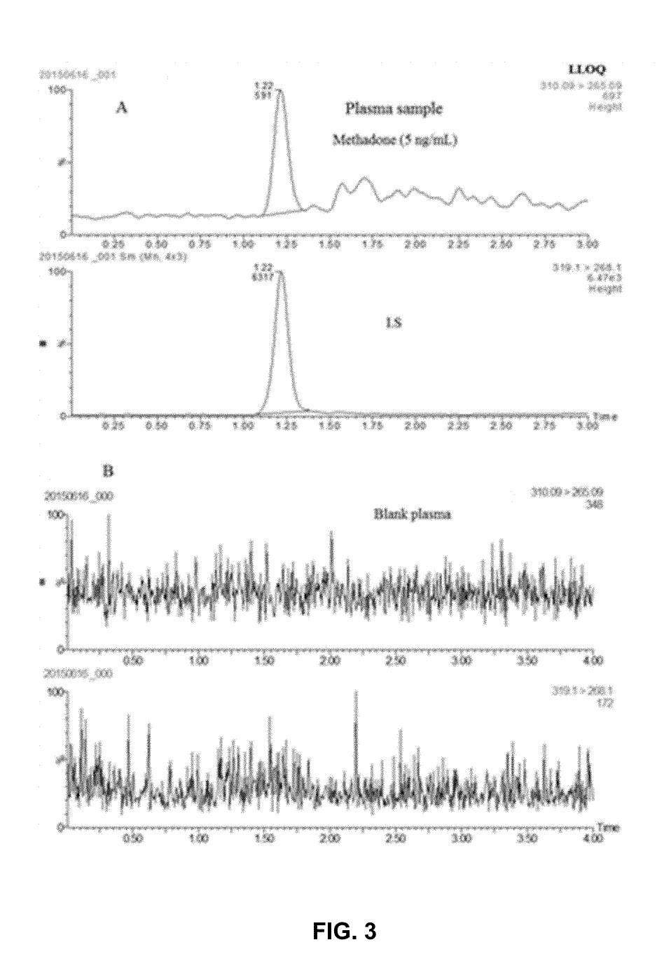

FIG. 3 shows a chromatogram for methadone in a plasma sample and blank plasma extracted by a tablet like that shown in FIGS. 1A and 1B. Generally, the data shows validation for determining methadone in plasma and amphetamine in urine. The methadone concentration in the plasma sample was 5 ng/mL, and the data in the figure shows good selectivity for the extraction of methadone from plasma using the tablets described above and below. The graphs show MRM transitions obtained from the analysis of methadone at LLOQ with internal standard (A) and blank plasma sample (B).

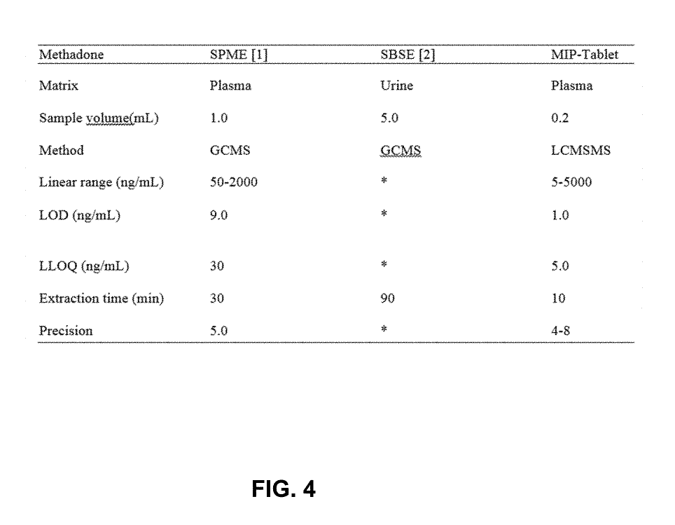

FIG. 4 is a table that compares LOD, LLOQ extraction time and accuracy for different solid-phase extraction techniques. In general, the comparison sets the MIP-Tablet described herein with published results for SPME and SBSE techniques.

The data shown here indicates that the MIP-Tablet technique considerably reduced the extraction time compared to SPME (decreased by three-fold) and SBSE (decreased by nine-fold). In addition, the sample volume for performing the operations was reduced by 5 times and 25 times as compared to using SPME and SBSE respectively.

The sample sizes for the different methods varies because it is largely dictated by the selected method. For example, SBSE requires relatively large sample volumes compares to SPE and the tablet method discussed here. As a result, the latter methods can be used for smaller sample volumes such as 100-200 micro-liters and for large sample volumes, such as 1 mL, while SPME and SBSE may require volumes of about 1-5 mL.

The linear range in the table indicates the concentration levels at which a particular method can be used accurately. A higher linear range indicates that a method is suitable for lower and higher concentration levels of an analyte of interest in a sample.

The extraction time for the subject tablet method is faster than the other methods because a thing film of polymer results in faster analyte diffusion into and out of the tablet than with other methods, and faster equilibrium times.

Precision in this example is measured as RSD % of quality control samples. Quality control samples (QSC) are used at three concentration levels as recommended by relevant FDA guidelines. In SPME data shown here, one concentration level was used.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A solid-form sampling tablet, comprising a tablet formed of a polymer having applied to it a thin-film polymer and having a porosity sized to accept a solid form analyte of interest from a liquid sample and to hold the solid form analyte in an internal portion of the tablet.

2. The sampling tablet of claim 1, wherein the tablet is sized for oral introduction and holding by a human subject.

3. The sampling tablet of claim 1, wherein the tablet is in the form of a short cylinder.

4. The sampling tablet of claim 1, wherein the tablet is 1 cm or less in diameter, and 0.5 cm or less in height.

5. The sampling tablet of claim 1, wherein the tablet contains voids of the same size and shape as the solid form analyte to which the tablet is directed.

6. The sampling tablet of claim 5, wherein the tablet is formed by molecularly imprinting a polymer around a form of the analyte to which the tablet is directed.

7. The sampling tablet of claim 5, wherein the analyte to which the tablet is directed is selected from the group consisting of methadone and amphetamine.

8. A method of producing a biologic liquid sampling tablet, the method comprising:
molecularly imprinting a polymer over a matrix of an analyte of interest for biological testing; and
removing the matrix from the imprinted polymer to form a porous tablet,
wherein the tablet is sized to be inserted in an ampoule or human oral cavity.

9. The method of claim 8, further comprising:
inserting the porous tablet in a liquid sample containing the analyte of interest;
removing the tablet from the sample containing the analyte of interest; and
submitting analyte captured in the tablet for automated chemical analysis.

10. The method of claim 9, wherein the liquid sample is inside an oral cavity of a patient to be analyzed.

11. The method of claim 10, wherein the tablet is maintained in the oral cavity for a time determine to be sufficient to infuse the tablet with a testable amount of the analyte of interest.

12. The method of claim wherein submitting the analyte captured in the tablet comprising removing the analyte from the tablet by subjecting the tablet to a solvent appropriate to remove the analyte from the tablet.
